(19) United States
(12) Patent Application Publication
(10) Pub. No.: US 2026/0297022 A1
(43) Pub. Date: Oct. 1, 2026

(54) PROCESS FOR THE PREPARATION OF SULFAMOYLBENZOIC ACID DERIVATIVES

(71) Applicant: IAMA THERAPEUTICS S.R.L., Genova (IT)

(72) Inventors: Marco BORGOGNO, Genova (IT); Annalisa SAVARDI, Genova (IT); Andrea PATRICELLI MALIZIA, Genova (IT); Marco DE VIVO, Genova (IT); Laura CANCEDDA, Genova (IT); Tommaso ANGELINI, Verona (IT); Riccardo GARZELLI, Verona (IT)

(21) Appl. No.: 19/474,875

(22) PCT Filed: Apr. 17, 2024
(86) PCT No.: PCT/IB2024/053748

§ 371 Date: Oct. 13, 2025

(30) Foreign Application Priority Data
Apr. 18, 2023 (IT) 102023000007521

(51) Int. Cl. C07C 303/38; C07D 295/155; C07D 295/26; C07D 309/14

(52) U.S. Cl. CPC C07C 303/38; C07D 295/155; C07D 295/26; C07D 309/14; C07C 2601/08

(57) ABSTRACT

The present invention relates to a method for the preparation of derivatives of the sulfamoylbenzoic acid, in particular of the (3-(dimethylsulfamoyl)-4-(8, 8, 8-trifluorooctylamino) benzoic acid.

### CROSS-REFERENCE TO RELATED APPLICATIONS

This Patent Application claims priority from Italian Patent Application No. 102023000007521 filed on Apr. 18, 2023, the entire disclosure of which is incorporated herein by reference.

### TECHNICAL FIELD

The present invention relates to a method for the preparation of derivatives of the sulfamoylbenzoic acid, in particular of the (3-(dimethylsulfamoyl)-4-(8,8,8-trifluorooctylamino)benzoic acid.

### BACKGROUND OF THE INVENTION

3-(dimethylsulfamoyl)-4-(8,8,8-trifluorooctylamino)benzoic acid (1) is an NKCC1 inhibitor useful in the treatment of neurological disorders (WO2020202072, Savardi et al., Chem, 2020; Borgogno et al., J. Med. Chem. 2021; Savardi et al., 2023).

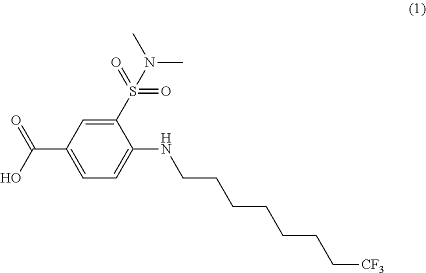 [SMILES: CN(C)S(=O)(=O)c1cc(C(=O)O)ccc1NCCCCCCCC(F)(F)F]

Patent WO 2020202072 describes a synthesis of the 3-(dimethylsulfamoyl)-4-(8,8,8-trifluorooctylamino)benzoic acid comprising the following steps:
1) conversion of the 3-(chlorosulfonyl)-4-fluorobenzoic acid (2) to the corresponding 3-dimethylsulfamoyl derivative (3)

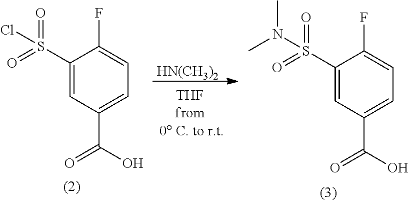

2) reaction of 8-bromo-1,1,1-trifluorooctane (4) with potassium phthalimide (5) to obtain 2-(8,8,8-trifluorooctyl)isoindoline-1,3-dione (6)

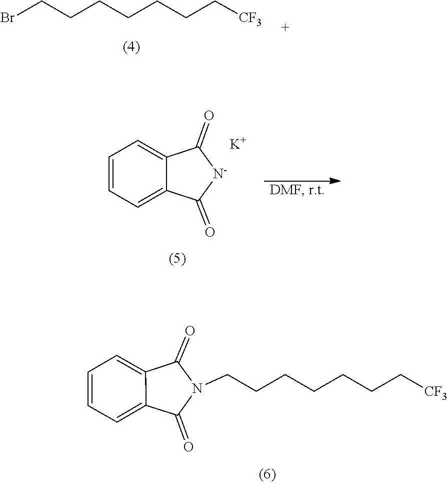 [SMILES: FC(F)(F)CCCCCCCBr ; O=C1[N-]C(=O)c2ccccc21 ; [K+] ; O=C1c2ccccc2C(=O)N1CCCCCCCC(F)(F)F]

3) deprotection of 8,8,8-trifluorooctane-1-amine (7) from the derivative (6) obtained in step (2)

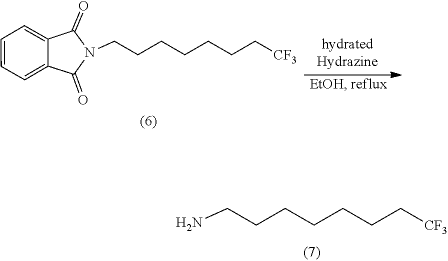 [SMILES: O=C1c2ccccc2C(=O)N1CCCCCCCC(F)(F)F ; NCCCCCCCC(F)(F)F]

4) reaction of the derivative (3) obtained in step (1) with the derivative (7) obtained in step (3) to provide the compound of formula (1).

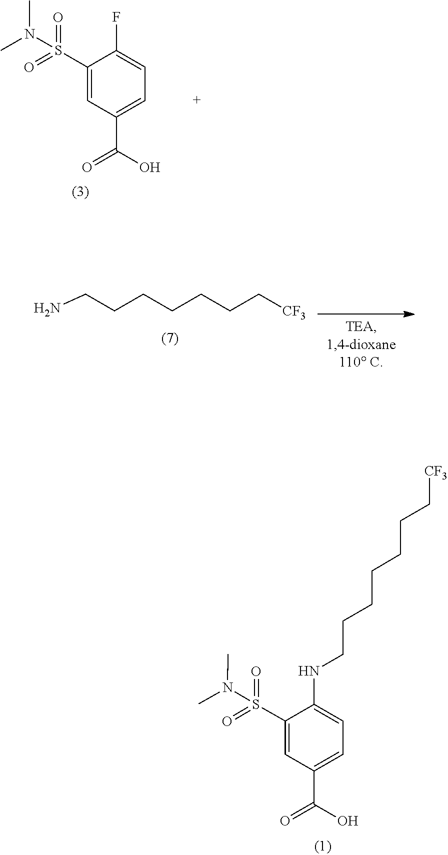 [SMILES: CN(C)S(=O)(=O)c1cc(C(=O)O)ccc1F ; NCCCCCCCC(F)(F)F ; CN(C)S(=O)(=O)c1cc(C(=O)O)ccc1NCCCCCCCC(F)(F)F]

The preparation process described in WO2020202072 is useful as a process for the preparation of sulfamoylbenzoic acid derivatives, but there is still much room for improvement in terms of total yield.

A further drawback of the synthesis described in WO2020202072 is the use of hazardous chemicals, in particular for the deprotection of the amine (7), which cannot be used for preparation on an industrial scale. Hydrazine is in fact a suspected carcinogen for humans. In the process of WO2020202072, several chromatographic purification steps are also used.

The need is therefore felt in the art for new methods for the preparation of sulfamoylbenzoic acid derivatives, without the disadvantages of the methods of the prior art.

Therefore, aim of the present invention is to provide a method for the synthesis of sulfamoylbenzoic acid derivatives with good total yield and suitable for use on an industrial scale.

### SUMMARY OF THE INVENTION

This aim is achieved thanks to the method of Claim 1.

### DESCRIPTION OF EMBODIMENTS

According to a first aspect of the invention there is provided a method for the preparation of a compound of formula (I) or its salts, pharmaceutically acceptable, stereoisomers, enantiomers, diastereoisomers, tautomers, geometric zwitterions:

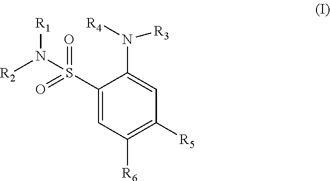

wherein:
R₁ and R₂ are, independently, H, substituted or unsubstituted, linear or branched C₁₋₁₀-alkyl optionally comprising one or more unsaturations; substituted or unsubstituted C₃₋₈-cycloalkyl; substituted or unsubstituted, linear or branched C₄₋₁₀-cycloalkyl-alkyl; C₃₋₈-heterocycle;
or, together with the nitrogen atom to which they are bound,
R₁ and R₂ form a substituted or unsubstituted saturated heterocycle;
R₃ and R₄ are, independently, hydrogen; substituted or unsubstituted C₁₋₁₀-alkyl optionally comprising one or more unsaturations; C₃₋₁₀-cycloalkyl; C₄₋₁₀-cycloalkyl-alkyl; C₂₋₈-haloalkyl; substituted or unsubstituted, linear or branched C₂₋₈-heteroalkyl; optionally substituted phenyl;
provided that at least one of R₃ and R₄ is other than hydrogen;
R₅ is hydrogen; halogen; hydroxyl; thiophenol; —NO₂;
R₆ is —COOH;
including the steps of:
   a) reacting, in aqueous solution, a compound of formula (II) with an amine of formula (III) to obtain a compound of formula (IV):

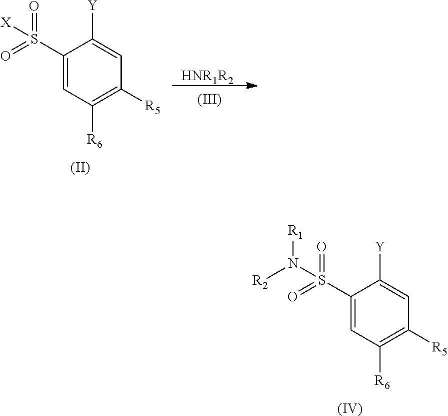

wherein X and Y are independently a halogen;
b) reacting, in aqueous solution and in the presence of a base, the compound of formula (IV) with a compound of formula (V) to obtain a compound of formula (I).

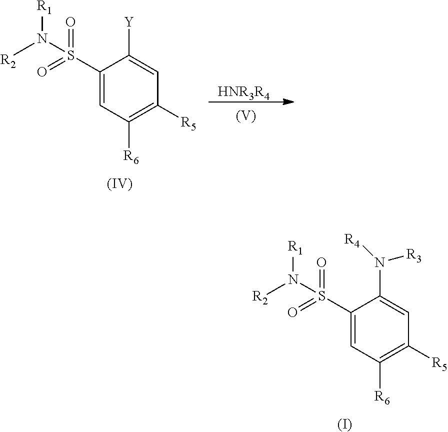

Step a) can be conducted at a temperature ranging between 0 and 20° C., preferably at 0° C.

Step b) may be conducted in the presence of an inorganic base selected from the group consisting of an inorganic phosphate and an inorganic carbonate, preferably selected from the group consisting of tripotassium phosphate and potassium carbonate, or of an organic base, preferably selected from the group consisting of N,N-diisopropylethylamine, triethylamine and trimethylamine.

According to one embodiment, the method of the invention when applied to obtain compounds of formula (I) wherein R₄=H, comprises, prior to step b), step a1) of reacting a compound of formula (VI) with a compound of formula (VII) to obtain a compound of formula (VIII) and subsequently reducing the obtained compound to obtain a compound of formula (V).

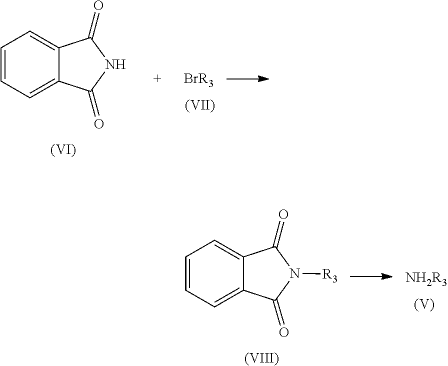

Step a1) may be conducted in the presence of a solvent selected from the group consisting of acetonitrile, acetone, isopropanol and methanol.

The reduction may be conducted in the presence of a reducing agent selected from the group consisting of sodium borhydride, sodium cyanoborohydride, lithium borohydride, lithium aluminum hydride, and diisobutylaluminum hydride.

The method of the invention can be used for the preparation of compounds of formula (I) wherein R₆ is COOH.

The method of the invention can be used for the preparation of compounds of formula (I) wherein R₅ is selected from the group consisting of H, OH, Cl and methoxyl.

The method of the invention can be used for the preparation of compounds of formula (I) wherein R₁ and R₂ are, independently, substituted or unsubstituted, linear or branched H, C₁₋₁₀-alkyl optionally comprising one or more unsaturations; substituted or unsubstituted C₃₋₈-cycloalkyl; substituted or unsubstituted, linear or branched C₄₋₁₀-cycloalkyl-alkyl; C₃₋₈-heterocycle; or, together with the nitrogen atom to which they are bound, R₁ and R₂ form a substituted or unsubstituted saturated heterocycle.

In one embodiment, R₁ and R₂ are selected from the group consisting of CH₃, H, morpholine, cyclopentyl and cyclohexyl.

The method of the invention can be used for the preparation of a compound of formula (I) selected from the group consisting of:
4-(butylamino)-3-(methylsulfamoyl)benzoic acid,
4-(hexylamino)-3-(methylsulfamoyl)benzoic acid,
3-(methylsulfamoyl)-4-(octylamino)benzoic acid,
4-(3,3-dimethylbutylamino)-3-(methylsulfamoyl)benzoic acid,
3-(methylsulfamoyl)-4-(8,8,8-trifluorooctylamino)benzoic acid,
4-(butylamino)-3-(dimethylsulfamoyl)benzoic acid,
3-(dimethylsulfamoyl)-4-(hexylamino)benzoic acid,
3-(dimethylsulfamoyl)-4-(octylamino)benzoic acid,
4-(3,3-dimethylbutylamino)-3-(dimethylsulfamoyl)benzoic acid,
3-(dimethylsulfamoyl)-4-(4,4,4 trifluorobutylamino)benzoic acid,
3-(dimethylsulfamoyl)-4-(6,6,6-trifluorohexylamino)benzoic acid,
3-(dimethylsulfamoyl)-4-(8,8,8-trifluorooctylamino)benzoic acid,
3-(dimethylsulfamoyl)-4-(2-methoxyethylamino)benzoic acid,
3-(dimethylsulfamoyl)-4-(4-methoxybutylamino)benzoic acid,
3-(dimethylsulfamoyl)-4-(6-methoxyhexylamino)benzoic acid,
3-(cyclopentylsulfamoyl)-4-(8,8,8-trifluorooctylamino)benzoic acid,
3-(cyclohexylsulfamoyl)-4-(8,8,8-trifluorooctylamino)benzoic acid,
5-(dimethylsulfamoyl)-2-hydroxy-4-(8,8,8-trifluorooctylamino)benzoic acid,
3-morpholinosulfonyl-4-(8,8,8-trifluorooctylamino)benzoic acid.

In particular the compound of formula (I) is 3-(dimethylsulfamoyl)-4-(8,8,8-trifluorooctylamino)benzoic acid.

In the following, the present invention will be shown by means of some examples, which are not intended to be considered limiting of the scope of the invention.

### Example 1

Preparation of the 3-(dimethylsulfamoyl)-4-(8,8,8-trifluorooctylamino)benzoic acid (1).

STEP 1) Preparation of the 3-(dimethylsulfamoyl)-4-fluoro-benzoic acid (INT-2). Aqueous N-dimethylamine (600 mL, 4.74 mol) was dissolved in water (600 mL). The solution was cooled to 0° C. and stirred. Then 3-chlorosulfonyl-4-fluoro-benzoic acid (300.00 g, 1.26 mol) was added in 5 portions of about 37.5 g each, under stirring for a total time of 82 minutes. After the last portion was added, it was verified that the reaction was complete (HPLC). Then 12 M hydrogen chloride (300 mL, 3.60 mol) was added dropwise at 0° C. for a total addition time of 45 min. During the addition, precipitation of the product was observed. The mixture was allowed to heat to 20° C. and the precipitated product was collected by filtration and washed with water (7×600 mL, 7×2 vol). The cake was completely de-liquored under high vacuum for 4 h and then dried in a vacuum oven for 48 h at 50° C.

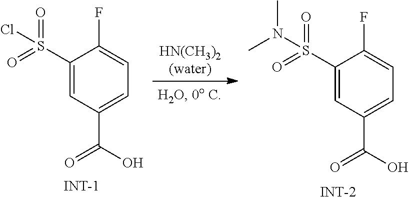

3-(Dimethylsulfamoyl)-4-fluoro-benzoic acid (INT-2) (299.0 g, 95% yield). 1H NMR (400 MHz, DMSO-d₆) δ 8.29-8.24 (m, 2H), 7.67-7.58 (m, 1H), 2.75 (d, J=1.9 Hz, 6H).

STEP 2) 8,8,8-trifluorooctane-1-amine (INT-7). 8-Bromo-1,1,1-trifluoro-octane (INT-4) (300.00 g, 1.21 mol) and (1,3-dioxoisoindolin-2-yl) potassium (INT-5) (337.31 g, 1.82 mol) were charged to a 2 L reactor along with acetonitrile (1500 mL, 5 vol). The mixture was stirred and refluxed at 110° C. overnight. Upon completion of the reaction (controlled by NMR), water (1500 mL, 5 vol) at room temperature was added to the mixture and transferred to a separatory funnel. The phases were separated and the inorganic phase was removed. The organic phase was evaporated at 1.5 vol (450 ml) and the resulting viscous liquid was transferred to a 10 L jacketed reactor with isopropanol (IPA) (2700 mL, 9 vol). Water (300 mL, 1 vol) was then added at 20° C. NaBH₄ (184.012 g, 4.86 mol) was added in 4 portions of 1 eq. each.

After the last addition, the mixture was heated and refluxed. After 1 hour of reflux the reaction reached the completion of the transformation into benzylalcoholic amide product (INT-8) (controlled by UPLC). The internal temperature was adjusted to 70° C. and water (1200 mL, 4 vol) was added dropwise over 20-25 minutes (maximum gas evolution: 0.3 m³/h). Once the gaseous evolution was attenuated, acetic acid (450 mL, 7.87 mol) was added slowly at the initial step of addition (maximum gaseous development kept at 0.8 m³/h) and increasing the speed after half the addition since the gaseous development ceased (addition time: 30-35 min). The mixture was then stirred at 70° C. all night. Upon completion of the reaction (controlled by UPLC) the reaction mixture was cooled to 20° C. and 11.6 M sodium hydroxide (1501 mL, 17.4 mol) (5 vol 35% NaOH) was added dropwise. The mixture was separated into phases and the organic phase was collected and diluted with methyl-t-butyl ether (MTBE) (1500 mL, 5 vol). The IPA/MTBE mixture was washed with 3 vol of 17.5% NaOH (35% solution dilution) to remove residual phthalide carboxylate (about 10 mol % at NMR). 12 M hydrogen chloride (152 mL, 1.82 mol) was then added dropwise leading to the formation of a turbidity (small sedimentation). The suspension was allowed to stir at 0° C. overnight. The solvent was evaporated under vacuum (115 mBar, jacket at 50° C.) up to a minimum stirrable volume (ca. 500-450 ml) and MTBE (3000 mL, 10 vol) was added. The suspension was allowed to stir at 0° C. (jacket temperature) overnight. The resulting precipitated solid was collected by filtration, transferred to a crystallization dish and dried overnight in a vacuum oven at 40° C.

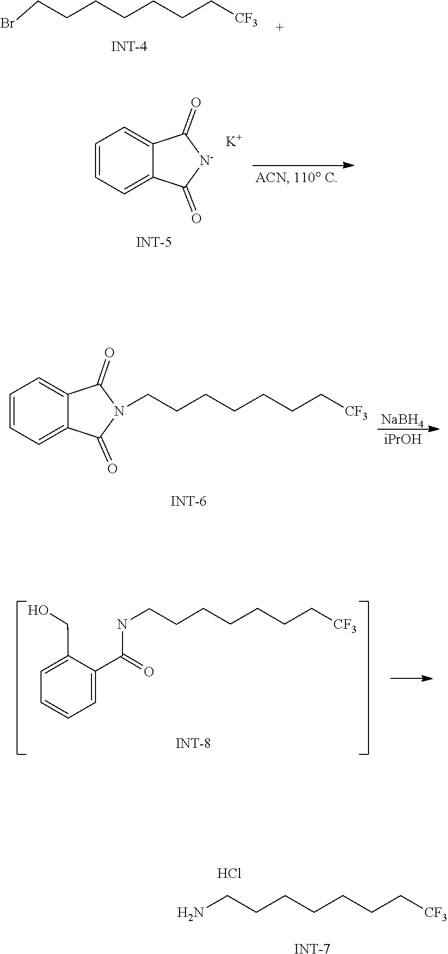

8,8,8-Trifluorooctylamine hydrochloride (INT-7) (237.0 g, 80% yield). 1H NMR (400 MHz, DMSO-d₆) δ 8.08 (s wide, 3H) 2.78-2.68 (m, 2H), 2.30-2.15 (m, 2H), 1.61-1.41 (m, 4H), 1.38-1.21 (m, 6H).

STEP 3) (3-(Dimethylsulfamoyl)-4-(8,8,8-trifluorooctylamino)benzoic acid (1). 3-(Dimethylsulfamoyl)-4-fluoro-benzoic acid (258.85 g, 1.05 mol), 8,8,8-trifluorooctan-1-amine hydrochloride (230.00 g, 1.05 mol) and tripotassium phosphate (888.95 g, 4.19 mol) were charged to a 2 L hydrogenation vessel. After addition of water (1150 mL, 5 vol), the vessel was sealed. The reaction mixture was stirred at 120° C. for 24 hours. Upon completion of the reaction (controlled by HPLC), the reaction mixture was diluted with water (1610 mL, 7 vol) after transfer to a 10 L JLR equipped with condenser. The mixture was heated to internal 95-100° C. and 12 M hydrochloric acid (349 mL, 4.19 mol) was added dropwise over 10-15 min. At the end of the addition the pH was 6/7 and the product as a solid separated upon reaching 20° C. The suspension was subjected to hot/cold treatment (over 1 h at 90° C., kept at 90° C. for 1 h, over 20 min at 40° C., kept at 40° C. for 2 h, over 4 h at 20° C.). The product was filtered and the cake was washed with water (3×690 mL, 3×3 vol). The filtered solid was then transferred to a vacuum oven overnight at 50° C.

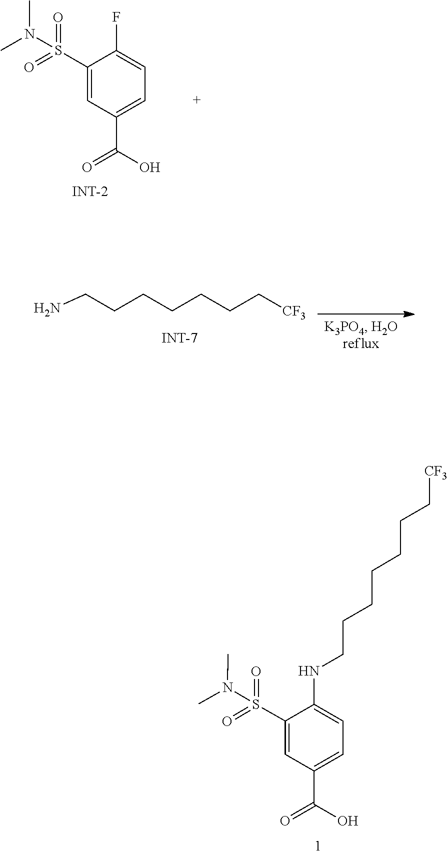

(3-(Dimethylsulfamoyl)-4-(8,8,8-trifluorooctylamino)benzoic acid (3.17) (98.0% w/w qNMR, 382.6 g, 87.3% yield). 1H NMR (400 MHz, DMSO-d₆) δ 12.62 (s, 1H), 8.05 (d, J=2.1 Hz, 1H), 7.93 (dd, J=8.8, 2.1 Hz, 1H), 6.91 (d, J=9.0 Hz, 1H), 6.75 (t, J=5.0 Hz, 1H) 0.75 (t, J=5.4 Hz, 1H), 3.24 (q, J=6.6 Hz, 2H), 2.29-2.14 (m, 2H), 1.64-1.52 (m, 2H), 1.52-1.39 (m, 2H), 1.40-1.25 (m, 6H).

STEP 4) Crystallization of the (3-(dimethylsulfamoyl)-4-(8,8,8-trifluorooctylamino)benzoic acid. The crude matter (3.17) (382.6 g, 0.93 mmol, 1 weight unit) was dissolved in 2-Me THF (5.5 Vol) at 20° C. The mixture was heated to 40° C. and filtered through a polish filter (0.45 μm); the filter was washed with another 2-Me THF (0.5 Vol).

The temperature was set to 20° C., then n-heptane (1.5 vol.) was added over 15 minutes. The mixture was seeded with compound (1) (0.7 wt % seed) and the mixture was stirred for 1 hour. Over 100 minutes additional n-heptane (2.5 Vol) was added and the mixture was allowed to mature for 20 minutes. Over 40 minutes additional n-heptane (2.0 vol.) was added and the mixture was subjected to 40 minutes ageing. Finally, n-heptane (5.1 vol.) was added over 60 minutes and the amalgam was allowed to mature for 30 minutes. The mixture was then filtered and the solid in the filter was washed with a mixture of 2-Me THF/n-heptane (25:75, 1 vol). Finally, the product was dried under vacuum at room temperature (307.64 g, yield 80%).

### Example 2

Aqueous kinetic solubility of derivative compounds of the benzoic acid starting from a solution of 10 mM DMSO in phosphate buffered saline (PBS) at pH 7.4

Aliquots of 30 μL of stock solution of the test compound in 10 mM DMSO were incubated in phosphate buffered saline (PBS) at pH 7.4 at 25° C. for 24 hours. After centrifugation, the compound dissolved in the supernatant was quantified by LC-MS/MS.

The target concentration was 250 μM with a final concentration of 2.5% DMSO. Aqueous kinetic solubility was determined by UV quantification at 215 nm. The kinetic solubility was then calculated by dividing the peak area of the supernatant by the peak area of the reference and multiplying by the reference concentration (μM) and the dilution factor (1.25).

As shown in Table 1, the compounds of formula (I) have a solubility ranging from 188 μM to greater than 250 μM, indicating a good aqueous solubility and a solubility comparable to that of compound 1, the synthesis of which is exemplified in Example 1.

TABLE 1

| | | Kinetic |
|---|---|---|
| Com- | | sol. PBS |
| pound | Structure | pH 7.4 |
| 1 | 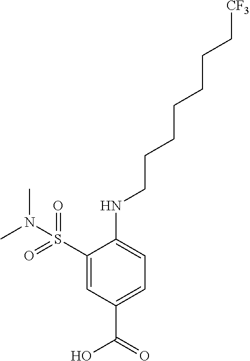 [SMILES: CN(C)S(=O)(=O)c1cc(C(=O)O)ccc1NCCCCCCCC(F)(F)F] | >250 μM |
| 2 | 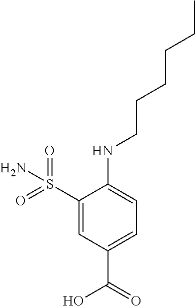 [SMILES: CCCCCCNc1ccc(C(=O)O)cc1S(N)(=O)=O] | 238 μM |
| 3 | 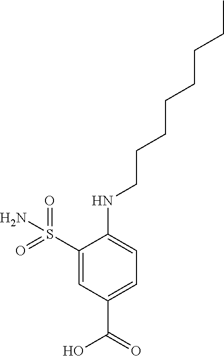 [SMILES: CCCCCCCCNc1ccc(C(=O)O)cc1S(N)(=O)=O] | >250 μM |
| 4 | 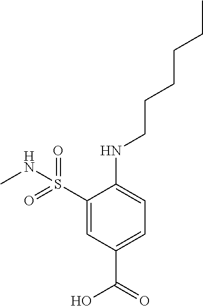 [SMILES: CCCCCCNc1ccc(C(=O)O)cc1S(=O)(=O)NC] | 247 μM |
| 5 | 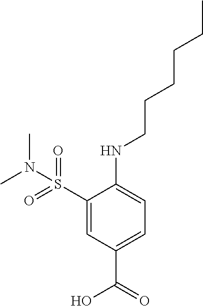 [SMILES: CCCCCCNc1ccc(C(=O)O)cc1S(=O)(=O)N(C)C] | 243 μM |
| 6 | 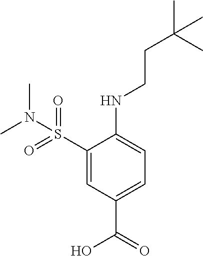 [SMILES: CN(C)S(=O)(=O)c1cc(C(=O)O)ccc1NCCC(C)(C)C] | >250 μM |
| 7 | 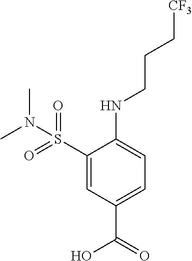 [SMILES: CN(C)S(=O)(=O)c1cc(C(=O)O)ccc1NCCCC(F)(F)F] | 245 μM |
| 8 | 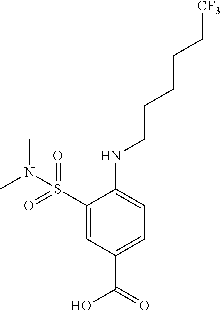 [SMILES: CN(C)S(=O)(=O)c1cc(C(=O)O)ccc1NCCCCCC(F)(F)F] | 249 μM |
| 9 | 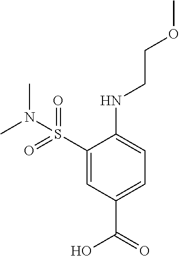 | 237 μM |
| 10 | 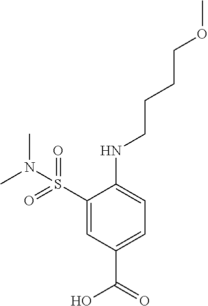 | 247 μM |
| 11 | 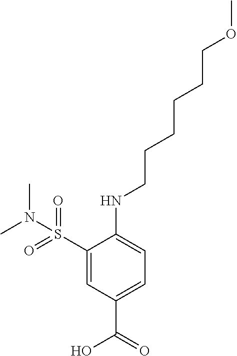 | >250 μM |
| 12 | 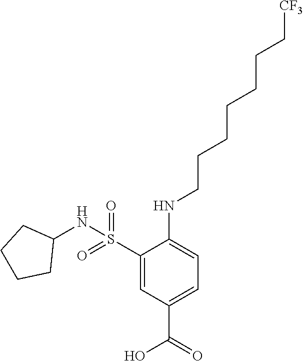 [SMILES: O=C(O)c1ccc(NCCCCCCCC(F)(F)F)c(S(=O)(=O)NC2CCCC2)c1] | 223 μM |
| 13 | 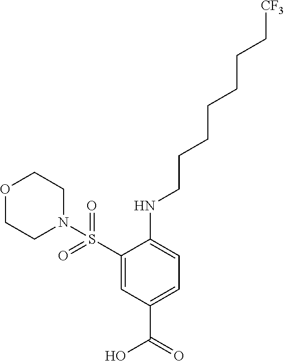 [SMILES: O=C(O)c1ccc(NCCCCCCCC(F)(F)F)c(S(=O)(=O)N2CCOCC2)c1] | 244 μM |
| 14 | 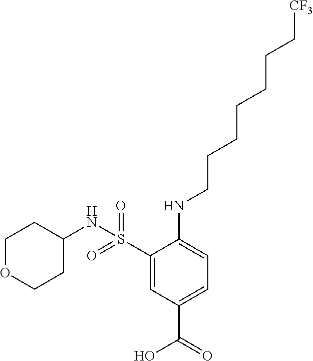 [SMILES: O=C(O)c1ccc(NCCCCCCCC(F)(F)F)c(S(=O)(=O)NC2CCOCC2)c1] | 244 μM |
| 15 | 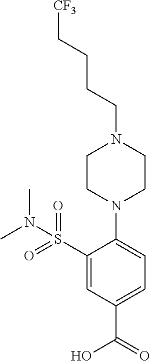 [SMILES: CN(C)S(=O)(=O)c1cc(C(=O)O)ccc1N1CCN(CCCCC(F)(F)F)CC1] | >250 μM |
| 16 | 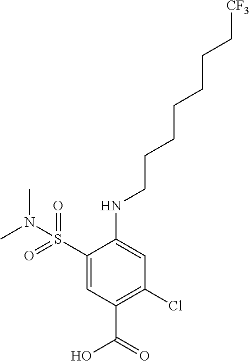 [SMILES: CN(C)S(=O)(=O)c1cc(C(=O)O)c(Cl)cc1NCCCCCCCC(F)(F)F] | 241 μM |
| 17 | 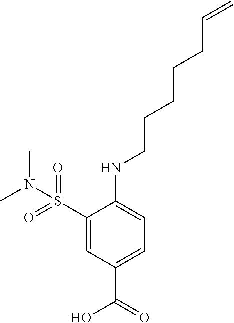 [SMILES: C=CCCCCCNc1ccc(C(=O)O)cc1S(=O)(=O)N(C)C] | >250 μM |
| 18 | 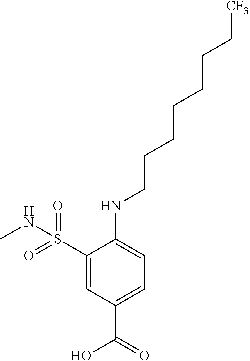 [SMILES: CNS(=O)(=O)c1cc(C(=O)O)ccc1NCCCCCCCC(F)(F)F] | 242 μM |
| 19 | 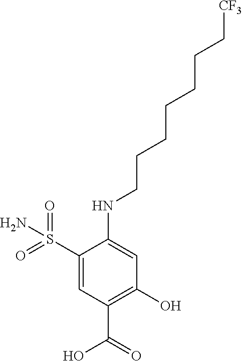 [SMILES: NS(=O)(=O)c1cc(C(=O)O)c(O)cc1NCCCCCCCC(F)(F)F] | >250 μM |
| 20 | 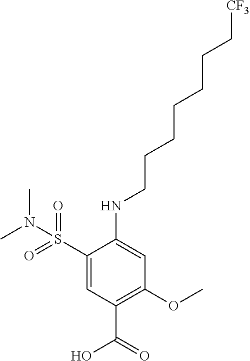 [SMILES: COc1cc(NCCCCCCCC(F)(F)F)c(S(=O)(=O)N(C)C)cc1C(=O)O] | 188 μM |

1. A method for the preparation of a compound of formula (I) or its salts, pharmaceutically acceptable stereoisomers, enantiomers, diastereoisomers, tautomers, geometric zwitterions: 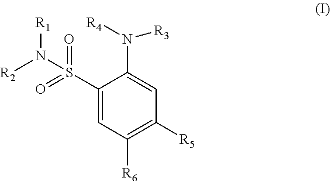
wherein:
R₁ and R₂ are, independently, H, substituted or unsubstituted, linear or branched C₁₋₁₀-alkyl optionally comprising one or more unsaturations; substituted or unsubstituted C₃₋₈-cycloalkyl; substituted or unsubstituted, linear or branched C₄₋₁₀-cycloalkyl-alkyl; C₃₋₈-heterocycle; or, together with the nitrogen atom to which they are bound, R₁ and R₂ form a substituted or unsubstituted saturated heterocycle;
R₃ and R₄ are, independently, hydrogen; substituted or unsubstituted C₁₋₁₀-alkyl optionally comprising one or more unsaturations; C₃₋₁₀-cycloalkyl; C₄₋₁₀-cycloalkyl-alkyl; C₂₋₈-haloalkyl; substituted or unsubstituted, linear or branched C₂₋₈-heteroalkyl; optionally substituted phenyl;
provided that at least one of R₃ and R₄ is other than hydrogen;
R₅ is hydrogen; halogen; hydroxyl; thiophenol; —NO₂;
R₆ is —COOH;
including the steps of:
a) reacting, in aqueous solution, a compound of formula (II) with an amine of formula (III) to obtain a compound of formula (IV): 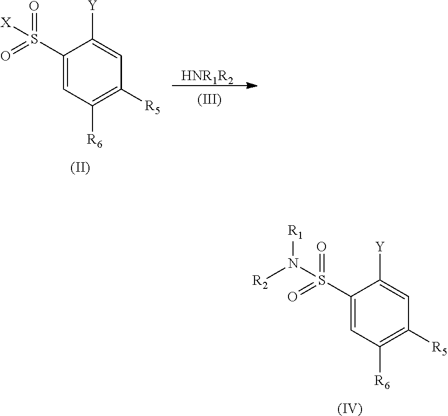
wherein X and Y are independently a halogen;
b) reacting, in aqueous solution and in the presence of a base, the compound of formula (IV) with a compound of formula (V) to obtain a compound of formula (I)

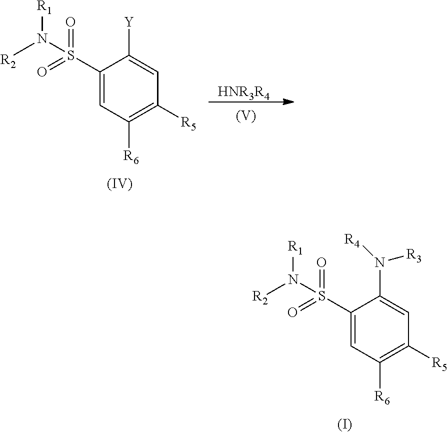

2. The method according to claim 1, wherein step b) is conducted in the presence of a base selected from the group consisting of an inorganic phosphate, an inorganic carbonate, N,N-diisopropylethylamine, triethylamine, and trimethylamine.

3. The method according to claim 2, wherein said base is selected from the group consisting of tripotassium phosphate and potassium carbonate.

4. The method according to claim 1 wherein R₄=H, characterized in that it comprises, prior to step b), step a1) of reacting a compound of formula (VI) with a compound of formula (VII) to obtain a compound of formula (VIII) and subsequently reducing the obtained compound to obtain a compound of formula (V)

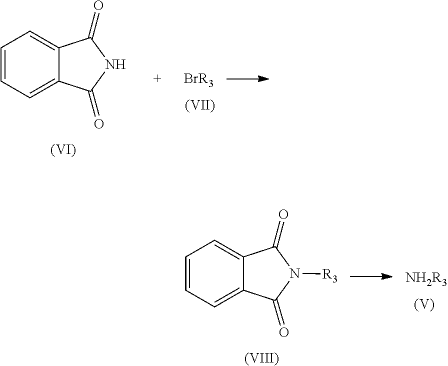

5. The method according to claim 4, wherein step a1) is conducted in a solvent selected from the group consisting of acetonitrile, acetone, isopropanol and methanol.

6. The method according to claim 4, wherein the reduction is conducted in the presence of a reducing agent selected from the group consisting of sodium borhydride, sodium cyanoborohydride, lithium borohydride, lithium aluminum hydride, and diisobutylaluminum hydride.

7. The method according to claim 1, wherein said R₅ is selected from the group consisting of H, OH, Cl, and methoxyl.

8. The method according to am claim 1, wherein said R₁ and R₂ are selected from the group consisting of CH₃, H, morpholine, cyclopentyl, and cyclohexyl.

9. The method according to claim 1, wherein said compound of formula (I) is selected from the group consisting of
4-(butylamino)-3-(methylsulfamoyl)benzoic acid,
4-(hexylamino)-3-(methylsulfamoyl)benzoic acid,
3-(methylsulfamoyl)-4-(octylamino)benzoic acid,
4-(3,3-dimethylbutylamino)-3-(methylsulfamoyl)benzoic acid,
3-(methylsulfamoyl)-4-(8,8,8-trifluorooctylamino)benzoic acid,
4-(butylamino)-3-(dimethylsulfamoyl)benzoic acid,
3-(dimethylsulfamoyl)-4-(hexylamino)benzoic acid,
3-(dimethylsulfamoyl)-4-(octylamino)benzoic acid,
4-(3,3-dimethylbutylamino)-3-(dimethylsulfamoyl)benzoic acid,
3-(dimethylsulfamoyl)-4-(4,4,4 trifluorobutylamino)benzoic acid,
3-(dimethylsulfamoyl)-4-(6,6,6-trifluorohexylamino)benzoic acid,
3-(dimethylsulfamoyl)-4-(8,8,8-trifluorooctylamino)benzoic acid,
3-(dimethylsulfamoyl)-4-(2-methoxyethylamino)benzoic acid,
3-(dimethylsulfamoyl)-4-(4-methoxybutylamino)benzoic acid,
3-(dimethylsulfamoyl)-4-(6-methoxyhexylamino)benzoic acid,
3-(cyclopentylsulfamoyl)-4-(8,8,8-trifluorooctylamino)benzoic acid,
3-(cyclohexylsulfamoyl)-4-(8,8,8-trifluorooctylamino)benzoic acid,
5-(dimethylsulfamoyl)-2-hydroxy-4-(8,8,8-trifluorooctylamino)benzoic acid, and
3-morpholinosulfonyl-4-(8,8,8-trifluorooctylamino)benzoic acid.

10. The method according to claim 9, wherein said compound of formula (I) is 3-(dimethylsulfamoyl)-4-(8,8,8-trifluorooctylamino)benzoic acid (1).
